(19) United States
(12) Patent Application Publication
(10) Pub. No.: US 2025/0132027 A1
(43) Pub. Date: Apr. 24, 2025

(54) HUMAN-MACHINE INTERACTION FOR CONTROLLING AND/OR CONFIGURING A MEDICAL TECHNOLOGY SYSTEM

(71) Applicant: Siemens Healthineers AG, Forchheim (DE)

(72) Inventors: Fabiola Fernández-Gutiérrez, Nürnberg (DE); Philipp Roser, Erlangen (DE)

(21) Appl. No.: 18/920,977

(22) Filed: Oct. 20, 2024

(30) Foreign Application Priority Data
Oct. 20, 2023 (EP) 23204786.0

(51) Int. Cl. G16H 40/60; G05B 13/02; G05B 13/04

(52) U.S. Cl. CPC G16H 40/60; G05B 13/027; G05B 13/04

(57) ABSTRACT

For human-machine interaction for controlling and/or configuring a medical technology system, a user input for a central computer-implemented language model (CIS) that relates to a performance of a medical examination and/or intervention using the medical technology system is captured. Using the central CIS, a first prompt for a first CIS is generated as a function of the user input. Using the first CIS, first instructions for controlling and/or configuring a first medical technology apparatus of the medical technology system are generated as a function of the first prompt. As a function of the first instructions, the first apparatus is at least partially automatically controlled and/or configured, and/or as a function of the first instructions, user information for controlling and/or configuring the first apparatus are output.

This application claims the benefit of European Patent Application No. EP 23204786.0, filed on Oct. 20, 2023, which is hereby incorporated by reference in its entirety.

### BACKGROUND

The present embodiments relate to human-machine interaction for controlling and/or configuring a medical technology system.

Medical technology apparatuses, for example imaging modalities such as ultrasound imaging systems, X-ray-based imaging systems and magnetic resonance tomography systems, C-arm devices, movable or adjustable patient couches, or apparatuses for performing therapeutic procedures, for instance HIFU systems (HIFU: “High-Intensity Focused Ultrasound”) or histotripsy systems, are often very complex and may have a large number of functions.

To be able to use such medical technology apparatuses appropriately, not only are a basic medical training and training specific to the medical technology apparatus in question often necessary, but also to a large extent practice and practical experience. Furthermore, it may be necessary to keep the respective knowledge about the workflows and functions up to date at all times.

Consequently this may mean that medical technology apparatuses are not operated correctly and/or that optimum use is not being made of their functionally.

This problem is intensified in the case of medical technology systems with multiple medical technology apparatuses and/or other apparatuses. For example, in imaging-assisted interventions there may be a series of apparatuses and apparatus components with which the medical personnel interacts, for example an angiography system, table control panels, display devices, EKG signaling devices, databases for retrieving the patient's medical history, etc. These apparatuses require a variety of user inputs, for example by hand or by foot via a pedal. At the same time apparatuses may be connected to one another and may exchange information with one another, which for example is likewise done manually. Thus for example patient information originating from an IT system of the hospital may be sent to the angiography system.

A further problem is that the individual apparatuses do not necessarily have standardized interfaces via which the exchange of information could be automated.

Computer-implemented language models, CIS, in particular so-called large language models (LLMs), for instance the GPT-4 described in the publication “GPT-4 Technical Report” (arXiv: 2303.08774v3), may be used as virtual assistants.

Such CISs are for example based on transformer network architectures of artificial neural networks that have been trained on the basis of large quantities of training data. The training data may in this case be generic or may be tailored to particular fields of application, also referred to as domains, or a combination of both approaches may be followed, wherein for example a generic CIS is refined by domain-specific further training for a specific field of application. Transformer networks were for example introduced in the publication A. Vaswani et al.: “Attention Is All You Need” (ArXiv: 1706.03762v7).

One challenge with the use of CIS is the efficient and correct formulation of user inputs (“prompting”), so that the CIS also in fact supplies an appropriate and usable output.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method for human-machine interaction for controlling and/or configuring a medical technology system in which a user may be more reliably supported, also in the case of complex medical technology systems, is provided.

The present embodiments are based on the idea of providing a central CIS with which a human user may interact, and of using the central CIS in order, based on a corresponding user input, to generate a prompt for a further CIS, which then in turn generates instructions for controlling and/or configuring an apparatus of the medical technology system assigned to the first CIS.

In accordance with one aspect of the present embodiments, a computer-implemented method for human-machine interaction for controlling and/or configuring a medical technology system is specified. In this case a user input for a central computer-implemented language model, CIS, is captured, in particular by means of a data processing apparatus via a user input interface. The user input relates to a performance of a medical examination and/or intervention using the medical technology system, in particular using a first apparatus of the medical technology system.

Using the central CIS, a first prompt for a first CIS assigned to the first apparatus of the medical technology system is generated as a function of the user input. Using the first CIS, first instructions for controlling and/or configuring the first apparatus are generated as a function of the first prompt. As a function of the first instructions the first apparatus is controlled and/or configured at least partially automatically and/or as a function of the first instructions user information for controlling and/or configuring the first apparatus are output, in particular by means of a user output interface of the data processing apparatus.

Unless specified otherwise, all steps of the computer-implemented method may be performed by the data processing apparatus, which has at least one computing unit. In particular, the at least one computing unit is configured or adapted to perform the steps of the computer-implemented method. To this end, the at least one computing unit may for example store a computer program, which contains commands which when executed by the at least one computing unit cause the at least one computing unit to execute the computer-implemented method.

A medical technology system may be a technical system for the, in particular at least partially automatic, performance of a diagnostic, therapeutic, surgical and/or imaging procedure. However, the performance of a diagnostic, therapeutic, and/or surgical procedure is generally not part of the computer-implemented method of the present embodiments.

The first apparatus may be a medical technology apparatus, which may be used directly for performing the diagnostic, therapeutic, surgical and/or imaging procedure, for example an X-ray-based angiography system, in particular a C-arm device, a computed tomography system, a magnetic resonance tomography system, an ultrasound imaging system, a HIFU system, a histotripsy system, etc. However, the first apparatus may also be used indirectly for the diagnostic, therapeutic, surgical and/or imaging procedure or for supporting the same. It may for example be a patient couch, an infrastructure apparatus, for instance a ventilation system or a lighting system or a display apparatus, etc.

A CIS is for example an LLM. A CIS may in particular be provided trained, i.e. it may be a language model that is known per se. The CIS may also be provided untrained and may be trained in a manner that is known per se on the basis of training data. It is also possible for the CIS to be provided pretrained and to be refined on the basis of further training data, for example via a correspondingly provided interface for programming applications (application programming interface, API).

The user input may for example be made in text form or otherwise as voice input, for example followed by a voice-to-text conversion. The first prompt for the first CIS is in particular generated in text form.

The user input may for example name or identify one or more members of the clinical team and/or name or identify a patient and/or specify a measure, in particular a diagnostic and/or therapeutic and/or surgical measure, to be performed by means of the medical technology system, in particular using the first apparatus.

The central CIS and the first CIS are in particular different from one another, i.e. are trained on the basis of in any case partially different training data. However, the underlying network architecture may be the same for the central CIS and the first CIS. The first CIS is in particular a domain-specific CIS, wherein the domain is given by the control and/or configuration of the first apparatus. The central CIS is in particular designed or trained on broader relationships relating to the entire medical technology system. Although it may likewise be regarded as a domain-specific CIS, the domain relates here not specifically to the first apparatus but to the control and/or configuration of the medical technology system, as well as where appropriate to clinical operational sequences that may be implemented with the medical technology system. In the following, the term domain-specific CIS hence does not include the central CIS, unless expressly stated otherwise.

For example, the central CIS may be trained on clinical workflows, derived from national and international clinical guidelines, clinic-specific standard work instructions, and/or historical settings.

The first CIS as a domain-specific CIS is for example trained on domain-specific information relating to the specific first apparatus or comparable apparatuses for the same purpose, for example on user manuals, earlier settings, procedures originating from publications by a manufacturer of the first apparatus or based on technical contents of marketing material, scientific journals, etc.

The fact that the first apparatus is at least partially automatically controlled and/or configured as a function of the first instructions may in particular be understood to mean that using the data processing apparatus the corresponding control commands and/or configurations may be transmitted directly to the first apparatus or to control modules of the first apparatus, so that at least part of the control and/or configuration of the first apparatus takes place without a further user input being necessary.

The user information for controlling and/or configuring the first apparatus, which is output as a function of the first instructions, serves in particular to instruct one or more members of the clinical team in the manual or partially manual control and/or configuration of the first apparatus.

The computer-implemented method may in particular be extended to medical technology systems, in which further apparatuses and correspondingly assigned domain-specific CIS are provided in addition to the first apparatus.

According to the computer-implemented method the central CIS acts as a universal interface to the user, whereas the first CIS, and where appropriate further domain-specific CISs, act as universal interfaces to the first apparatus or where appropriate to further apparatuses of the medical technology system. As a result, the control and/or configuration of the medical technology system is significantly simplified overall for the clinical team, and in particular the human-machine interaction with the medical technology system may be centralized. As a result, it is possible to dispense, at any rate in part, with standardized interfaces for the exchange of information between different components of the medical technology system.

According to at least one form of embodiment the first apparatus is a medical technology apparatus and in particular contains an imaging system.

Because of the high complexity and large number of different configuration and application options of medical technology apparatuses, in particular of imaging systems, the present embodiments have a particularly advantageous effect here.

For example, the first apparatus may be triggered on the basis of the first instructions to initiate or perform a data capture action or data capture sequence for imaging determined by the first instructions and/or to move one or more movable components of the first apparatus into a position and/or orientation determined by the first instructions or to guide them along a track determined by the first instructions, etc. For example, operating parameters of a radiation source, for example an X-ray radiation source, of the first apparatus and/or of a filter apparatus, for example an X-ray filter apparatus, of the first apparatus may be set on the basis of the first instructions, etc.

According to at least one form of embodiment the imaging system contains a C-arm X-ray imaging system, an angiography system, a computed tomography system, a magnetic resonance tomography system, a positron emission tomography system and/or an ultrasound imaging system.

Because of the high complexity and large number of different configuration and application options of such imaging systems, the present embodiments have a particularly advantageous effect here.

According to at least one form of embodiment, using the central CIS a second prompt for a second CIS, in particular a second domain-specific CIS, assigned to a second apparatus of the medical technology system, is generated as a function of the user input. Using the second CIS, second instructions for controlling and/or configuring the second apparatus of the medical technology system are generated as a function of the second prompt. As a function of the second instructions the second apparatus is at least partially automatically controlled and/or configured, and/or as a function of the second instructions user information for controlling and/or configuring the second apparatus is output, in particular by means of the user output interface of the data processing apparatus.

The above explanations regarding the first CIS or the first apparatus may be transferred analogously to the second CIS or the second apparatus. However, the second apparatus is generally different from the first apparatus and is for example not a medical technology apparatus in the narrower sense as described above. In particular, the first CIS and the second CIS are trained for different domains, i.e. in particular based on different training data.

The central CIS may thus here serve as a universal user interface for at least two different apparatuses of the medical technology system, without the clinical team having to create the associated inputs, also referred to as prompts, for the domain-specific CIS assigned to the apparatuses.

According to at least one form of embodiment the second apparatus contains a lighting apparatus, for example a lighting apparatus to illuminate a room, in particular a room in which the first apparatus is arranged.

Accordingly, on the basis of the second instructions the optimal lighting conditions for the measure to be performed and/or the patient and/or the clinical team may be achieved.

According to at least one form of embodiment, using the central CIS, first data, which contains configuration data for the first apparatus and/or patient data for the patient and/or operator data for an operative of the medical technology system, i.e. for example a member of the clinical team, may be retrieved from at least one database as a function of the user input. The first apparatus is at least partially automatically controlled and/or configured as a function of the first data, for example as a function of the first instructions and the first data, and/or the user information is output as a function of the first data, for example as a function of the first instructions and the first data.

The configuration data may for example contain or relate to possible adjustable parameters or parameter ranges or other operating ranges of one of the apparatuses of the medical system, etc. The patient data may for example contain or relate to personal data, for instance height, weight, sex, age, health status, progression of the disease, etc. The operator data may for example contain or relate to the identity of the operative, personal data, for instance the height, and/or the operative's preferred settings relating to one of the apparatuses of the medical system, etc.

In this case the central CIS may retrieve the configuration data, the patient data and/or the operator data from the at least one database directly, or otherwise indirectly, for example via a further domain-specific CIS.

Thus the performance of the examination or intervention may be further automated and optimized.

According to at least one form of embodiment, using the central CIS, a third prompt for a third CIS is generated as a function of the user input. Using the third CIS, the first data is retrieved from the at least one database as a function of the third prompt.

The third CIS in this case retrieves the first data in particular directly from the at least one database. On the basis of the third prompt the third CIS may in particular generate instructions for the retrieval of the first data from the at least one database.

Thus the performance of the examination or intervention may be further automated and optimized.

According to at least one form of embodiment, using the central CIS, a fourth prompt for a fourth CIS, in particular a fourth domain-specific CIS, assigned to a fourth apparatus of the medical technology system, is generated as a function of the user input. Using the fourth CIS, fourth instructions for controlling and/or configuring the fourth apparatus of the medical technology system are generated as a function of the fourth prompt. As a function of the fourth instructions the fourth apparatus is at least partially automatically controlled and/or configured and/or user information for controlling and/or configuring the fourth apparatus is output as a function of the fourth instructions, in particular by means of the user output interface of the data processing apparatus.

The above explanations regarding the first, second and third CIS may be transferred analogously to the fourth CIS or the fourth apparatus.

According to at least one form of embodiment the fourth apparatus contains a patient table, for example a patient table for use with the first apparatus.

For example, the patient table may be at least partially automatically moved and/or adjusted in accordance with two or more axes of rotation and/or axes of translation. The fourth instructions in particular relate to the movement of the patient table in accordance with the two or more axes of rotation and/or axes of translation.

Accordingly, on the basis of the second instructions the optimal settings of the patient table, for example its height and/or orientation and/or position in the room, etc., for the measure to be performed and/or the patient and/or the clinical team may be achieved.

According to at least one form of embodiment, using the central CIS, a fifth prompt for a fifth CIS, in particular a fifth domain-specific CIS, assigned to a fifth apparatus of the medical technology system may be generated as a function of the user input. Using the fifth CIS, fifth instructions for controlling and/or configuring the fifth apparatus of the medical technology system are generated as a function of the fifth prompt. As a function of the fifth instructions the fifth apparatus is at least partially automatically controlled and/or configured and/or user information for controlling and/or configuring the fifth apparatus is output as a function of the fifth instructions, in particular by means of the user output interface of the data processing apparatus.

The above explanations regarding the first, second, third and fourth CIS may be transferred analogously to the fifth CIS or the fifth apparatus.

According to at least one form of embodiment the fifth apparatus contains a display device, in particular one or more monitors.

Accordingly, on the basis of the fifth instructions the optimal settings of the display device for the measure to be performed and/or the clinical team, for example its orientation and/or position in the room, a screen layout, a selection of data to be displayed, etc., are achieved.

According to at least one form of embodiment a further user input for the central CIS is captured, which contains a status query relating to vital parameters of the patient and/or a status, in particular a current status, of the first apparatus. Using the central CIS, a further first prompt for the first CIS is generated as a function of the further user input. Using the first CIS, further first instructions for the retrieval of the patient's vital parameters and/or of the status of the first apparatus are generated as a function of the further first prompt. As a function of the further first instructions the patient's vital parameters and/or the status of the first apparatus are retrieved at least partially automatically.

The explanations regarding the user input may be transferred analogously to the further user input. The explanations relating to the first prompt and the first instructions may be transferred analogously to the further first prompt or the further first instructions.

The patient's vital parameters may for example contain a blood pressure, a pulse rate, a respiratory rate, a blood oxygen saturation, EEG data, EKG data, etc. The patient's vital parameters are in particular retrieved by one or more vital parameter sensor apparatuses. The status of the first apparatus may for example contain a current or planned position and/or orientation of one or more movable components of the first apparatus, one or more currently set or planned operating parameters of the first apparatus, etc. The status of the first apparatus is in particular retrieved from the first apparatus.

Thus the performance of the examination or intervention may be further automated and optimized.

Depending on whether the medical system contains the second apparatus, the at least one database, the fourth apparatus and/or the fifth apparatus, the status query may in some forms of embodiment, alternatively or additionally to the status of the first apparatus, relate to a corresponding status of the second apparatus, the at least one database, the fourth apparatus or the fifth apparatus. The explanations on the status of the first apparatus may where appropriate be transferred analogously.

According to at least one form of embodiment the user input is a text input and/or the further user input is a text input.

The central CIS may hence directly process the user input and/or the further user input.

According to at least one form of embodiment the user input is a voice input. The voice input is converted into a text input, in particular by means of a voice-to-text software module of the data processing apparatus, and the first prompt is generated as a function of the text input.

The user may hence effect the user input by voice.

According to at least one form of embodiment the further user input is a further voice input. The further voice input is converted into a further text input, in particular by means of the voice-to-text software module, and the further first input is generated as a function of the further text input.

According to at least one form of embodiment the central CIS is trained on the basis of training data.

According to at least one form of embodiment the training data contains specified clinical guidelines and/or examination protocols and/or intervention protocols for the first apparatus and/or scientific publications relating to examinations and/or interventions that may be performed using the medical technology system.

In this way the central CIS may be effectively trained at a high level for its task as a central user interface.

According to at least one form of embodiment the training data contains historic user inputs for the central CIS and/or historic configuration data for the medical technology system.

In this way the central CIS may be effectively trained at a high level for its task as a central user interface.

According to at least one form of embodiment the first CIS is trained on the basis of further training data.

According to at least one form of embodiment the further training data contains at least one user manual and/or at least one set of operating instructions and/or at least one description for the first apparatus.

In this way the first CIS may be effectively trained domain-specifically.

According to at least one form of embodiment the further training data contains specified clinical guidelines and/or examination protocols and/or intervention protocols for the first apparatus and/or scientific publications relating to examinations and/or interventions that may be performed using the first apparatus.

In this way the first CIS may be effectively trained domain-specifically.

According to at least one form of embodiment the further training data contains historic configuration data and/or control data for the first apparatus.

In this way the first CIS may be effectively trained domain-specifically.

According to at least one form of embodiment the second CIS and/or the third CIS and/or the fourth CIS and/or the fifth CIS are trained on the basis of respective training data. The explanations and forms of embodiment relating to the further training data for training the first CIS may be transferred analogously to the respective training data for training the second CIS, the third CIS, the fourth CIS or the fifth CIS.

According to at least one form of embodiment the central CIS contains a trained artificial neural network, which is designed as a transformer network and/or the first CIS contains a further trained artificial neural network, which is designed as a transformer network.

Such neural networks, for instance the GPT-4 mentioned in the introduction, have proven to be particularly efficient.

According to at least one form of embodiment the second CIS and/or the third CIS and/or the fourth CIS and/or the fifth CIS each contain a corresponding trained artificial neural network, which is designed as a transformer network.

For individual applications or application situations that may arise in a computer-implemented method of the present embodiments and that are not explicitly described herein, it may be provided that, according to the method, an error message and/or a request to input user feedback is output and/or a default setting and/or a predetermined initial status is set.

According to a further aspect of the present embodiments, a data processing apparatus, having at least one computing unit, is specified. The at least one computing unit is designed to perform a computer-implemented method of the present embodiments.

A computing unit may in particular be understood as a data processing device which contains a processing circuit. The computing unit may thus in particular process data for the performance of computing operations. Where appropriate, this also includes operations in order to perform indexed accesses to a data structure, for example a look-up table (LUT).

The computing unit may in particular contain one or more computers, one or more microcontrollers and/or one or more integrated circuits, for example one or more application-specific integrated circuits (ASIC), one or more field-programmable gate arrays (FPGA), and/or one or more systems on a chip (SoC). The computing unit may also contain one or more processors, for example one or more microprocessors, one or more central processing units (CPU), one or more graphics processing units (GPU) and/or one or more signal processors, in particular one or more digital signal processors (DSP). The computing unit may also contain a physical or a virtual group of computers or other of the above-mentioned units.

In different example embodiments the computing unit contains one or more hardware and/or software interfaces and/or one or more storage units.

A storage unit may be designed as a volatile data memory, for example as a dynamic random access memory (DRAM) or a static random access memory (SRAM), or as a non-volatile data memory, for example as a read-only memory (ROM), as a programmable read-only memory (PROM), as an erasable programmable read-only memory (EPROM), as an electrically erasable programmable read-only memory (EEPROM), as a flash memory or flash EEPROM, as a ferroelectric random access memory (FRAM), as a magnetoresistive random access memory (MRAM) or as a phase-change random access memory (PCRAM).

According to at least one form of embodiment of the data processing apparatus the at least one computing unit stores the central CIS and the first CIS.

According to at least one form of embodiment of the data processing apparatus the at least one computing unit stores the second CIS and/or the third CIS and/or the fourth CIS and/or the fifth CIS.

According to a further aspect of the present embodiments, a medical technology system is specified which has the data processing apparatus and the first apparatus.

According to at least one form of embodiment of the medical technology system the medical technology system contains the second apparatus and/or the at least one database and/or the fourth apparatus and/or the fifth apparatus.

Further forms of embodiment of the medical technology system follow directly from the various configurations of the computer-implemented method and vice versa. In particular, individual features and corresponding explanations along with advantages regarding the various forms of embodiment may be transferred to the computer-implemented method of the present embodiments analogously to corresponding forms of embodiment of the medical technology system.

According to a further aspect of the present embodiments, a computer program containing commands is specified. When the commands are executed by a data processing apparatus (e.g., a data processing apparatus of the present embodiments), the commands cause the data processing apparatus to perform a computer-implemented method of the present embodiments.

The commands may for example be present as program code. The program code may for example be provided as binary code or assembler and/or as source code of a programming language, for example C, and/or as a program script, for example Python.

According to a further aspect of the present embodiments, a computer-readable storage medium is specified, which stores a computer program of the present embodiments.

The computer program and the computer-readable storage medium are each computer program products containing the commands.

Further features and combinations of features of the present embodiments emerge from the figures and the description thereof, and from the claims. In particular, further forms of embodiment need not necessarily contain all the features of one of the claims. Further forms of embodiment may have features or combinations of features that are not mentioned in the claims.

The present embodiments are explained in greater detail below on the basis of specific example embodiments and associated schematic drawings. In the figures the identical or functionally identical elements may be provided with the same reference characters. The description of identical or functionally identical elements is where appropriate not necessarily repeated in respect of different figures.

### BRIEF DESCRIPTION OF THE DRAWINGS

FIG. 1 shows a schematic representation of an example form of embodiment a medical technology system;

FIG. 2 shows a schematic flow diagram of an example form of embodiment of a computer-implemented method for human-machine interaction for controlling and/or configuring a medical technology system; and

FIG. 3 shows a schematic representation of a further example form of embodiment of a medical technology system.

### DETAILED DESCRIPTION

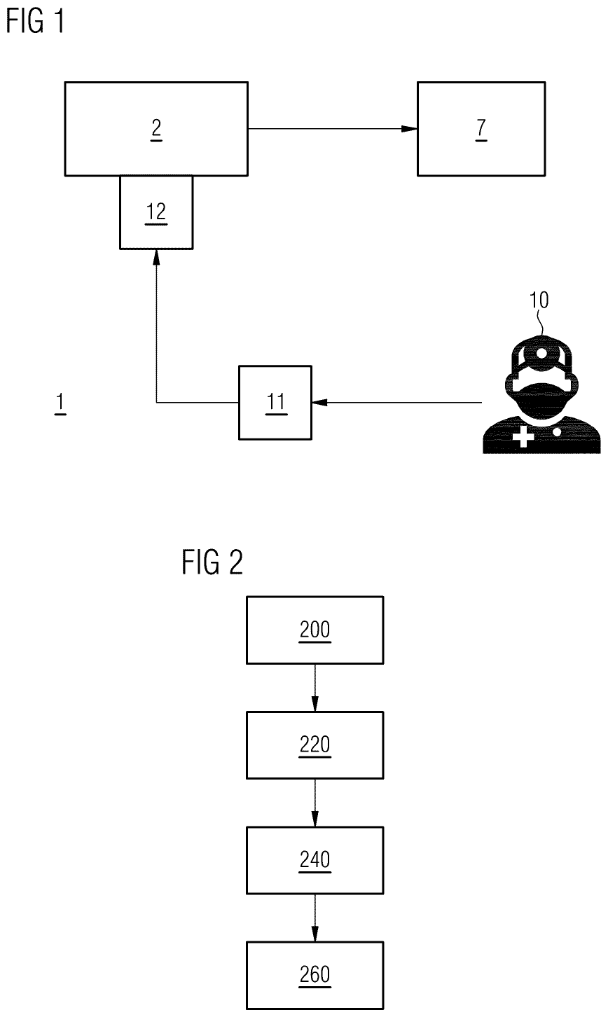

FIG. 1 schematically shows an example form of embodiment of a medical technology system 1.

The medical technology system 1 has an embodiment of a data processing apparatus with at least one computing unit, which is adapted to perform a computer-implemented method of the present embodiments for human-machine interaction for controlling and/or configuring the medical technology system 1. The medical technology system 1 additionally has a first apparatus 2, for example an imaging system. The at least one computing unit stores a central CIS 11 and a first CIS 12 assigned to the first apparatus 2.

FIG. 2 shows a schematic flow diagram of an example form of embodiment of a computer-implemented method for human-machine interaction for controlling and/or configuring the medical technology system 1 in FIG. 1.

In step 200 the at least one computing unit captures a user input of a user 10 for the central CIS 11 via a user input interface, for example as a text input or as a voice input. The user input relates to a performance of a medical examination and/or intervention by means of the medical technology system 1, in particular using the first apparatus 2.

In step 220, using the central CIS 11, as a function of the user input, where appropriate after conversion of the voice input into text, a first prompt for the first CIS 12 assigned to the first apparatus 2 is generated. In step 240, using the first CIS 12, as a function of the first prompt, first instructions for controlling and/or configuring the first apparatus 2 are generated.

In step 260, the first apparatus 2 is at least partially automatically controlled and/or configured as a function of the first instructions. Alternatively or additionally, user information for controlling and/or configuring the first apparatus 2 is output as a function of the first instructions, in particular via a user output apparatus 7, for example a display, of the medical technology system 1.

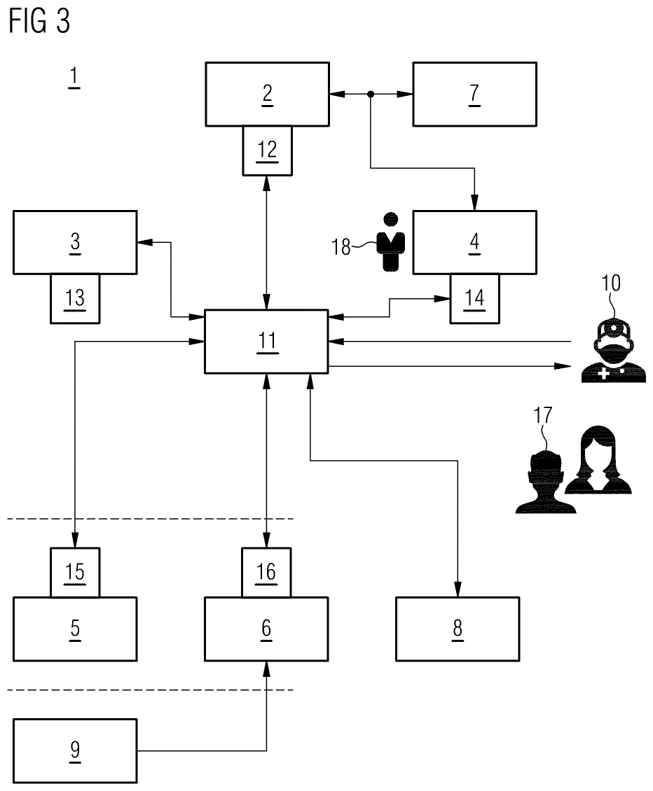

FIG. 3 schematically shows a further example form of embodiment of a medical technology system 1 that is based on the medical technology system 1 shown in FIG. 1.

The medical technology system 1 in accordance with FIG. 3 has, apart from the first apparatus 2, a second apparatus 3, for example a room lighting apparatus, and/or a third apparatus 4, for example a patient table. The medical technology system 1 accordingly also has a second CIS 13 assigned to the second apparatus 3 and/or a fourth CIS 14 assigned to the third apparatus 4.

Using the central CIS 11, a second prompt may be generated for the second CIS 13 and/or a third prompt for the third CIS 14 as a function of the user input. Using the second CIS 13, second instructions for controlling and/or configuring the second apparatus 3 may be generated as a function of the second prompt or third instructions for controlling and/or configuring the third apparatus 4 as a function of the third prompt. As a function of the second instructions the second apparatus 3 is for example at least partially automatically controlled and/or configured, thus in particular the room lighting is adjusted. Alternatively or additionally, the third apparatus 4 is for example at least partially automatically controlled and/or configured as a function of the third instructions, thus in particular the position and/or orientation of the patient table is set and/or its movement planned.

For example, the medical technology system 1 may have a database 5 relating to configurations of the first apparatus 2 and optionally a fourth CIS 15 assigned to the database 5.

Using the central CIS 11, configuration data for the first apparatus 2 may for example be retrieved from the database 5 as a function of the user input. To this end the central CIS 11 may access the database 5 directly or may generate a fourth prompt for the fourth CIS 15, which retrieves the configuration data from the database 5 as a function of the fourth prompt.

For example, the medical technology system 1 may have an interface 6 to an external database 9, and optionally a fifth CIS 16 assigned to the interface 6.

Using the central CIS 11, patient data for a patient 18 on whom the examination or intervention is to be performed may for example be retrieved from the database 9 as a function of the user input. To this end the central CIS 11 may directly access the interface 6 for the retrieval of the database 9 or may generate a fifth prompt for the fifth CIS 16, which retrieves the patient data from the database 9 via the interface 6 as a function of the fifth prompt.

For example, the medical technology system 1 may have a database 8. Using the central CIS 11, operator data for an operative 10, 17 of the medical technology system 1 may for example be retrieved from the database 8 as a function of the user input. To this end the central CIS may for example access the database 8 directly.

The operator data and/or the patient data and/or the configuration data may then for example be used at least partially automatically to control and/or configure the first apparatus 2.

In an example form of embodiment, the first apparatus 2 may be an X-ray-based angiography system, the second apparatus 3 the room lighting apparatus, and the third apparatus 4 the patient table.

In the user input, the user 10, for example a senior physician, may identify himself and the other members 17 of the clinical team, as well as the patient 18 and the examination or intervention to be performed.

The central CIS 11 may, on the basis of the user input, for example retrieve preferred settings relating to the room lighting apparatus, the patient table and/or the user output apparatus 7 from the database 8.

The central CIS 11 may, on the basis of the user input, generate the fifth prompt for the fifth CIS 16 and on the basis thereof this may retrieve the patient information from the database 9, in order to initiate the setting of the patient table and of the angiography system.

The central CIS 11 may, on the basis of the user input, generate the second prompt for the second CIS 13 and on the basis thereof this may generate instructions for the room lighting apparatus, in order to set the room lighting correspondingly.

The central CIS 11 may, on the basis of the user input, generate the third prompt for the third CIS 14 and on the basis thereof this may generate instructions for the patient table, in order to set the patient table correspondingly.

As described in particular with reference to the figures, the present embodiments enable a method for human-machine interaction for controlling and/or configuring a medical technology system to be implemented in which a user may be more reliably supported.

In different forms of embodiment the central CIS understands preferences and/or personal settings of members of a medical team, for example based on the history of examinations and/or interventions performed using the medical technology system or a comparable medical technology system.

In different forms of embodiment the stored history also contains method and/or patient features. In different forms of embodiment the central CIS may communicate with the clinical team by voice control and/or with further CISs, in particular the first and/or the second CIS, in a text-based fashion.

In different forms of embodiment the central CIS may decide which of the domain-specific CISs is to be employed or what information should be provided in order to start or continue the examination or intervention. If for example the user input does not contain enough information to perform the next pending step, the central CIS may request the corresponding information from the user, for example also as voice output.

In different forms of embodiment the central CIS may also act as an information broker between the domain-specific CISs. It may for example monitor which information is to be exchanged between the domain-specific CISs and for example react accordingly if the exchange of information between the domain-specific CISs contains incomplete or incorrect information.

In different forms of embodiment, multiple domain-specific CISs are provided, which have been trained for the respective domain. These CISs may be trained on the basis of device- and domain-specific information. For example, a CIS assigned to an angiography system could be trained on the basis of information, including corresponding user manuals, earlier settings used in hospitals, and proven methods originating from trustworthy sources, for example from the manufacturer of the angiography system or from renowned scientific journals. The domain-specific CISs monitor and control functions that may be controlled on the corresponding apparatus, in particular in dialog with the central CIS. If a request from the central CIS is not sufficient to fulfill the corresponding task, the respective domain-specific CIS also requests additional information from the central CIS.

In different forms of embodiment, each domain-specific CIS may register with the central CIS and for example provide specific information about its capabilities, its intended or defined purpose, etc. In this way the central CIS may establish which prompt should be transmitted to which domain-specific CIS.

In different forms of embodiment a user may also submit interdisciplinary queries, for example the range in which a patient's particular vital parameters lie, etc. The central CISs may then query the corresponding domain-specific CIS and for example compile an appropriate response for the user.

Independent of the grammatical term usage, individuals with male, female or other gender identities are included within the term. This includes in particular but not only the terms “patient”, “user”, and “physician”.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for human-machine interaction for controlling, configuring, or controlling and configuring a medical technology system, the method being computer-implemented and comprising:
capturing a user input for a central computer-implemented language model (CIS), which relates to a performance of a medical examination, intervention, or examination and intervention using the medical technology system;
generating, using the central CIS, a first prompt for a first CIS assigned to a first apparatus of the medical technology system as a function of the user input;
generating, using the first CIS, first instructions for controlling, configuring, or controlling and configuring the first apparatus as a function of the first prompt; and
automatically controlling, configuring, or controlling and configuring the first apparatus as a function of the first instructions, outputting user information for controlling, configuring, or controlling and configuring the first apparatus as a function of the first instructions, or a combination thereof.

2. The method of claim 1, wherein the first apparatus includes an imaging system.

3. The method of claim 2, wherein the imaging system includes a C-arm X-ray imaging system, an angiography system, a computed tomography system, a magnetic resonance tomography system, a positron emission tomography system, an ultrasound imaging system, or any combination thereof.

4. The method of claim 1, further comprising:
generating, using the central CIS, a second prompt for a second CIS assigned to a second apparatus of the medical technology system as a function of the user input;
generating, using the second CIS, second instructions for controlling, configuring, or controlling and configuring the second apparatus as a function of the second prompt; and
at least partially automatically controlling, configurating, or controlling and configuring the second apparatus as a function of the second instructions, outputting user information for controlling, configuring, or controlling and configuring the second apparatus as a function of the second instructions, or a combination thereof.

5. The method of claim 4, wherein the second apparatus includes a lighting apparatus, a patient table, a display device, or any combination thereof.

6. The method of claim 1, further comprising:
retrieving, using the central CIS, first data that contains configuration data for the first apparatus, patient data, operator data for an operative of the medical technology system, or any combination thereof from at least one database as a function of the user input; and
at least partially automatically controlling, configuring, or controlling and configuring the first apparatus as a function of the first data, outputting the user information as a function of the first data, or a combination thereof.

7. The method of claim 6, further comprising:
generating, using the central CIS, a third prompt for a third CIS as a function of the user input; and
retrieving, using the third CIS, the first data from the at least one database as a function of the third prompt.

8. The method of claim 1, further comprising:
capturing a further user input for the central CIS, the further user input including a status query relating to vital parameters for a patient, a status of the first apparatus, or the vital parameters for the patient and the status of the first apparatus;
generating, using the central CIS, a further first prompt for the first CIS as a function of the further user input;
generating, using the first CIS, further first instructions for retrieving the vital parameters for the patient, the status of the first apparatus, or the vital parameters for the patient and the status of the first apparatus as a function of the further first prompt; and
at least partially automatically retrieving the vital parameters for the patient, the status of the first apparatus, or the vital parameters for the patient and the status of the first apparatus as a function of the further first instructions.

9. The method of claim 1, wherein the user input is a text input, or
wherein the user input is a voice input, the voice input is converted into a text input, and the first prompt is generated as a function of the text input.

10. The method of claim 1, wherein the central CIS is trained based on training data, and wherein:
the training data contains specified clinical guidelines, examination protocols, intervention protocols, or any combination thereof for the medical technology system, scientific publications relating to examinations, interventions, or examinations and interventions that are performable using the medical technology system;
the training data contains historic user inputs for the central CIS, historic configuration data for the medical technology system, or a combination thereof; or
a combination thereof.

11. The method of claim 1, wherein the first CIS is trained based on further training data, and
wherein:
the further training data contains at least one user manual, at least one set of operating instructions, at least one description for the first apparatus, or any combination thereof;
the further training data contains specified clinical guidelines, examination protocols, intervention protocols for the first apparatus, scientific publications relating to examinations, interventions that are performable using the first apparatus, or any combination thereof;
the further training data contains historic configuration data, control data, or historic configuration data and control data for the first apparatus; or
any combination thereof.

12. The method of claim 1, wherein the central CIS includes a trained artificial neural network configured as a transformer network, the first CIS includes a further trained artificial neural network configured as a transformer network, or a combination thereof.

13. A data processing apparatus for human-machine interaction for controlling, configuring, or controlling and configuring a medical technology system, the data processing apparatus comprising:
a processor configured to:
capture a user input for a central computer-implemented language model (CIS), which relates to a performance of a medical examination, intervention, or examination and intervention using the medical technology system;
generate, using the central CIS, a first prompt for a first CIS assigned to a first apparatus of the medical technology system as a function of the user input;
generate, using the first CIS, first instructions for controlling, configuring, or controlling and configuring the first apparatus as a function of the first prompt; and
automatically control, configure, or control and configure the first apparatus as a function of the first instructions, output user information for controlling, configuring, or controlling and configuring the first apparatus as a function of the first instructions, or a combination thereof.

14. A medical technology system comprising:
a data processing apparatus for human-machine interaction for controlling, configuring, or controlling and configuring a medical technology system, the data processing apparatus comprising:
a processor configured to:
capture a user input for a central computer-implemented language model (CIS), which relates to a performance of a medical examination, intervention, or examination and intervention using the medical technology system;
generate, using the central CIS, a first prompt for a first CIS assigned to a first apparatus of the medical technology system as a function of the user input;
generate, using the first CIS, first instructions for controlling, configuring, or controlling and configuring the first apparatus as a function of the first prompt; and
automatically control, configure, or control and configure the first apparatus as a function of the first instructions, output user information for controlling, configuring, or controlling and configuring the first apparatus as a function of the first instructions, or a combination thereof; and
the first apparatus.

15. In a non-transitory computer-readable storage medium that stores instructions executable by one or more processors for human-machine interaction for controlling, configuring, or controlling and configuring a medical technology system, the instructions comprising:
capturing a user input for a central computer-implemented language model (CIS), which relates to a performance of a medical examination, intervention, or examination and intervention using the medical technology system;
generating, using the central CIS, a first prompt for a first CIS assigned to a first apparatus of the medical technology system as a function of the user input;
generating, using the first CIS, first instructions for controlling, configuring, or controlling and configuring the first apparatus as a function of the first prompt; and
automatically controlling, configuring, or controlling and configuring the first apparatus as a function of the first instructions, outputting user information for controlling, configuring, or controlling and configuring the first apparatus as a function of the first instructions, or a combination thereof.
